(19) United States
(12) Patent Application Publication
(10) Pub. No.: US 2023/0243815 A1
(43) Pub. Date: Aug. 3, 2023

(54) AN APPARATUS COMPRISING MICROFLUIDIC PROTEIN-BASED SENSORS AND METHODS OF USING SAME

(71) Applicant: UNIVERSITY OF MARYLAND, BALTIMORE COUNTY, Baltimore, MD (US)

(72) Inventors: Leah Tolosa CROUCHER, Baltimore, MD (US); Govind RAO, Baltimore, MD (US); Xudong GE, Baltimore, MD (US); Abhay ANDAR, Baltimore, MD (US); Hasibul HASAN, Baltimore, MD (US); Sheniqua BROWN, Baltimore, MD (US); Yordan KOSTOV, Baltimore, MD (US); Chad SUNDBERG, Baltimore, MD (US); Vikash KUMAR, Baltimore, MD (US); Shayan BORHANI, Baltimore, MD (US); Sarah BURNEY, Baltimore, MD (US); Samyukta RAO, Baltimore, MD (US)

(21) Appl. No.: 17/999,437

(22) PCT Filed: May 21, 2021
(86) PCT No.: PCT/US21/33571

§ 371 Date: Nov. 21, 2022

Related U.S. Application Data
(60) Provisional application No. 63/028,649, filed on May 22, 2020.

(51) Int. Cl. G01N 33/53; G01N 21/64; G01N 35/10; B01L 3/00; G01N 33/543

(52) U.S. Cl. CPC G01N 33/5308; G01N 21/6428; G01N 35/1097; B01L 3/502761; G01N 33/54386; G01N 2021/6439; B01L 2200/0647

(57) ABSTRACT

An automatic and continuous glucose monitoring apparatus and method of using same based on binding proteins. The apparatus integrates an aseptic sampling technique, a specially modified chromatography column to hold immobilized binding protein, and a microfluorometer into a compact portable device. The apparatus permits the measurement of a very wide range of concentrations—from a few micromolar to several hundreds of millimolar of glucose.

### CROSS REFERENCE TO RELATED APPLICATIONS

This application claims priority to U.S. Provisional Patent Application No. 63/028,649 filed on May 22, 2020 in the name of Leah Tolosa Croucher, Govind Rao, Xudong Ge, and Abhay Andar and entitled “Microfluidic Protein-Based Sensors,” which is hereby incorporated by reference herein in its entirety.

### FIELD

The present invention relates to a glucose monitoring apparatus and method of using same based on binding proteins. The apparatus and method of using same is capable of sample collection without consuming analyte, is highly portable, has a lower cost than systems of the prior art, is easy to use, and is capable of automatic and continuous sample collection and measurement.

### BACKGROUND OF THE INVENTION

Glucose is one of the most vital nutrients in the living organisms as it provides the energy necessary for different physiological activities in the organisms. Monitoring of glucose concentrations in mammals is necessary for proper diagnosis, treatment, and preventive cares. Similarly, glucose monitoring is one of the key requirements in bioprocesses such as microbial and mammalian cell cultures, fermentations etc. Most available glucose monitoring devices are electrochemical sensors which use direct probes or sample using a closed loop system. Disadvantageously, electrochemical sensors can introduce sterility issues to a cell culture, consume the analytes, consume the electrochemical used, and can be invasive in medical applications.

In the development of glucose sensors of submillimolar sensitivities, ligand-specific binding periplasmic proteins have been investigated for their potential use as optical biosensors [Kubo, 2002; Naal et al., 2002; Tian et al., 2007; Cuneo et al., 2006; Ge et al., 2003; Tolosa et al., 2003; Ge et al., 2007]. The binding proteins undergo conformational changes upon binding to their specific substrates. If a polarity sensitive fluorophore is introduced into the protein, the conformational change due to the binding to the substrate can be detected and this can be readily used as an optical sensor [Hellinga et al., 1998; Ribeiro et al., 2019]. Studies have been carried out that focus on the expression and purification of these biosensors [Ge et al., 2005; Ge et al., 2004] and many others have developed sampling techniques and functioning devices for glucose concentration measurement [Tolosa et al., 1999; Ge et al., 2008; Kostov et al., 2014; Sardesai et al., 2015: Tiangco et al., 2016; Saxl et al., 2011: Brown, 2019]. However, most devices of the prior art use separate components for sampling and fluorescence measurement which are often bulky, expensive, and suitable only for single measurements. Furthermore, the sample collection followed by the measurement of fluorescence using these devices has also been difficult to automate.

There is a continued need to develop a glucose monitoring apparatus and method of using same that is capable of sample collection without consuming analyte, is highly portable, has a lower cost, is easy to use, and is capable of automatic and continuous sample collection and measurement.

### SUMMARY OF THE INVENTION

In one aspect, the present invention relates to an apparatus for measuring the concentration of a substrate, said apparatus comprising:
a sampling device;
a microfluidic column device comprising a microfluidic column which comprises a solid support comprising immobilized binding protein, wherein the sampling device is communicatively connected to the microfluidic column; and
a fluorometer.

In another aspect, the present invention relates to a method of measuring the concentration of a substrate in a sample solution, said method comprising:
(a) inserting a sampling device of an apparatus into a sample comprising said substrate;
(b) flowing buffer through the sampling device at a flow rate, wherein the substrate diffuses from the sample into the buffer to yield a substrate-containing buffer;
(c) introducing the substrate-containing buffer to a microfluidic column in a microfluidic column device, wherein the substrate binds to a binding protein and an increase in fluorescence occurs; and
(d) measuring the fluorescence using a fluorometer,
wherein the microfluidic column device comprising the microfluidic column which comprises a solid support comprising immobilized binding protein, wherein the sampling device is communicatively connected to the microfluidic column.

Other aspects, features and embodiments of the invention will be more fully apparent from the ensuing disclosure and appended claims.

### BRIEF DESCRIPTION OF THE FIGURES

FIG. 1(A) is a top view of the designed microfluidic column (on the left). The inlet frit can be seen inside the inlet. A bottom view of the microfluidic column is shown on the right, showing the immobilized GBP is inside the channel.

FIG. 1(B) is a photograph of a microdialysis device for collecting glucose from the sample.

FIG. 2(A) is a schematic of an automatic glucose monitoring system described herein, comprising a syringe pump, valves, a microdialysis device and a sensor. The sensor is a microfluidic column that can be positioned over a microfluorometer.

FIG. 2(B) shows an embodiment of the glucose monitoring device showing the microdialysis device immersed into the sample.

FIG. 3 is a schematic of the control circuit showing the communication of the components to the computer.

FIG. 4 illustrates the fluorescence response during a single measurement cycle for a 40 mM glucose sample with a sampling flow rate of 2 mL/min. The binding and unbinding of the glucose to the GBP can be visualized from the fluorescence response.

FIG. 5 illustrates the fluorescence for different concentrations in consecutive cycles.

FIG. 6 illustrates the fluorescence amplitude corresponding to the glucose concentration of the sample solution in three experiments having the same sampling flow rate of 2 mL/min.

FIG. 7 illustrates the fluorescence response and slopes for different sampling flow rates, 2.5 mL/min (top) and 800 μL/min (bottom).

FIG. 8(A) illustrates the reading from the fiber optic fluorometer when a QBP biosensor was immobilized in a microfluidic column and alternating PBS and varying glutamine concentrations were introduced.

FIG. 8(B) illustrates the linear relationship of normalized intensity signal change (ΔF) relative to glutamine concentration.

FIG. 8(C) illustrates the linear relationship of the corresponding slope with glutamine concentrations.

FIG. 9(A) illustrates amino acids that bind at varying affinities to a microfluidic column biosensor, where F/F0 is the fluorescence intensity ratio.

FIG. 9(B) illustrates amino acids can be detected separately over the length of a microfluidic column.

FIG. 10 is a photograph of a microfluorimeter.

### DETAILED DESCRIPTION, AND PREFERRED EMBODIMENTS THEREOF

An apparatus for automatic and continuous substrate monitoring based on binding proteins, and a method of using same, is described herein. Broadly, the apparatus integrates an aseptic sampling technique, a modified chromatography column to hold immobilized binding protein, and utilizes a fluorometer for fluorescence measurement. A robust control circuit to integrate all the components and a computer program can be developed to automate all the functions. The method of using the device allows measurement of a very wide range of substrate concentrations, from a few micromolar to several hundreds of millimolar. In a preferred embodiment, the substrate comprises glucose and the binding protein is a glucose binding protein (GBP).

In one aspect, an apparatus for measuring the concentration of a substrate is described, said apparatus comprising:
a sampling device;
a microfluidic column device comprising a microfluidic column which comprises a solid support comprising immobilized binding protein, wherein the sampling device is communicatively connected to the microfluidic column; and
a fluorometer.
   The apparatus can further comprise at least one of: a syringe pump to circulate fluid within the apparatus; at least one valve to control the flow direction of the fluid within the apparatus; a check valve; a control circuit for communication between a computer and electrical components in the apparatus; tubing for communicative connections; a computer program; a computer; and any combination thereof, as described herein.

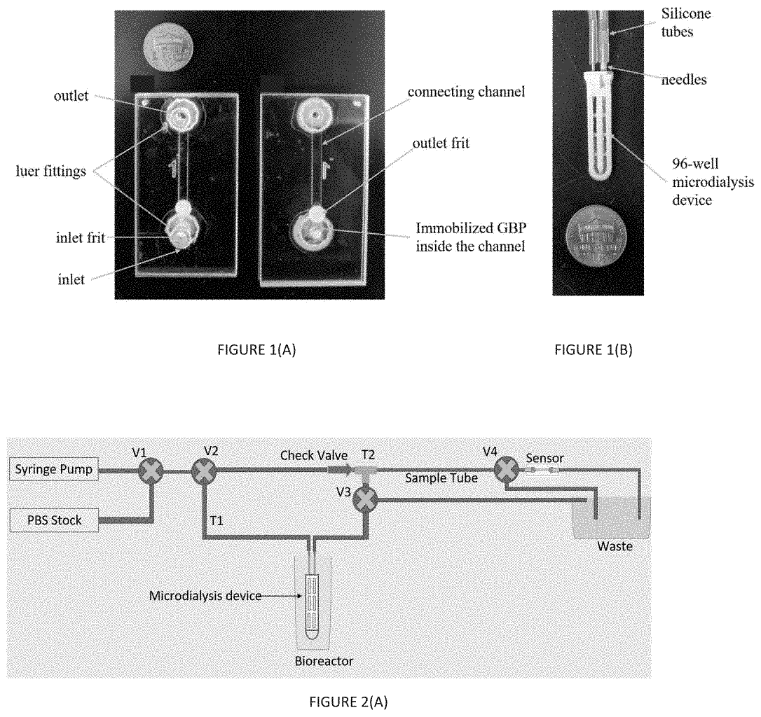

Modified chromatography columns are preferably versatile, customizable, robust, low-cost, and easily manufacturable. In a preferred embodiment, the modified chromatography column, hereinafter a “microfluidic column,” has a volume of about 0.1 to about 200 μL and accommodates any solid support. A microfluidic column device comprising a microfluidic column can be easily manufactured using equipment and solvent bonding methods available at most biochemical research laboratories. In one embodiment, the microfluidic column devices comprise layers of polymeric sheets, fittings such as caps and plugs (e.g., fuer fillings), and frits (see, for example, FIG. 1), as previously described by Andar et al. [Andar et al., 2019]. For example, the polymeric sheets can comprise polymethylinethacrylate (PMMA), poly dimethyl sulfoxane, polyethylene, polycarbonate, cellulose or wood among others, and a specific design for the microfluidic column, having the volume defined herein, can be printed on the PMMA sheets using laser printing or other techniques known in the art. The layers can be bonded, for example, using solvent bonding using ethanol. The fittings, such as luer lock fittings, hose barbs, ferrules, directly and/or welded tubes, can be affixed to the microfluidic column device using glue or other affixation means such as heat sealing. It should be appreciated by the person skilled in the art that this description of a microfluidic column device is not intended to limit same, and can be manufactured out of other materials, using other printing (e.g., 3D printing) and bonding methods, and include other fittings so long as the microfluidic column device comprises a microfluidic column having the volume defined herein, the microfluidic column can comprise a solid support (e.g., resin) of choice, has fittings that can be communicatively connected to the tubing of the sampling device, and is inert to the materials that are going to be introduced to the microfluidic columns including, but not limited to, fluids, proteins, glucose, and resins.

The microfluidic column devices comprise a solid support, wherein a binding protein is immobilized on said support. The glucose to be measured should be known to readily bind to the binding protein, thus exposing the fluorophore. Binding proteins may be immobilized on the solid support by any method including, but not limited to, physical adsorption, by ionic or covalent bond formation, or combinations thereof. In one embodiment, the binding protein is immobilized on the support via a tag including, but not limited to, a histidine tag (also referred to as a polyhistidine tag), streptavidin-biotin, glatathione S-transferase (GST), FLAG tag (Sigma-Aldrich), and Small Ubiquitin-like Modifier (SUMO). Other tags are readily determined by the person skilled in the art depending on the solid support and the binding protein used. A solid support may comprise polymers, glass, or metal. A solid support may comprise natural or synthetic materials. A solid support may comprise organic polymers such as polystyrene, polyethylene, polypropylene, polyfluoroethylene, polyethyleneoxy, polyacrylamide, and cellulose, as well as co-polymers and grafts thereof. A support may also be inorganic, such as glass, silica, controlled-pore-glass (CPG), or reverse-phase silica. The configuration of a solid support may be in the form of beads, spheres, particles, granules, a gel, or a surface., typically of sizes ranging from about 10-500 microns.

In one embodiment, the solid support comprises a resin that is fluorescently labeled with the binding protein of choice prior to introduction to the microfluidic column. Following labeling, the resin comprising the immobilized protein is introduced to the microfluidic column, utilizing frits to maintain the labeled resin in a preferred location in the microfluidic column. In another embodiment, the solid support comprises a resin that is introduced to the column prior to labeling the resin with the binding protein, again utilizing frits to maintain the resin in a preferred location in the microfluidic column. Frits preferably comprise inert materials such as polytetrafluoroethylene (PTFE), polysulfone, and/or cellulose acetate. Resins chosen should be known to be readily labeled with the binding protein of choice and can be eluted, for example using a buffer such as PBS. Chromatography resins are well known in the art and are contemplated for use in the device herein including, but are not limited to, Superflow Ni-NTA (Qiagen), Talon Cellthru Cobalt (Clontech), and/or Ni-NTA agarose (Thermo). In a preferred embodiment, the solid support comprises a resin that is introduced to the column prior to fluorescently labeling the resin with the binding protein. The packed and bound microfluidic columns yield substantially similar results if manufactured and packed identically. However, slight deviations might occur which is taken care of by calibration.

It should be appreciated by the person skilled in the art that the microfluidic column device can comprise one or more microfluidic columns, wherein when there are two or more microfluidic columns, they are arranged in parallel or in series. For example, a microfluidic column device can comprise two or more microfluidic columns that are not communicatively connected, arranged such that each microfluidic column has its own dedicated fluorimeter or one fluorometer having multiple fluorescence measuring channels. This allows the simultaneous measurement of more than one sample. Another example is the serial or parallel arrangement of microfluidic columns, which are communicatively connected, wherein each microfluidic column has a different binding protein immobilized therein. This permits the measurement of more than one substrate from the same sample. In still another alternative, each unique binding protein further comprises a unique dye, and the microfluidic column comprises two or more immobilized binding protein/dye combinations. A sample comprising two or more substrates (specific to the binding proteins) can then be separated based on spectral responses.

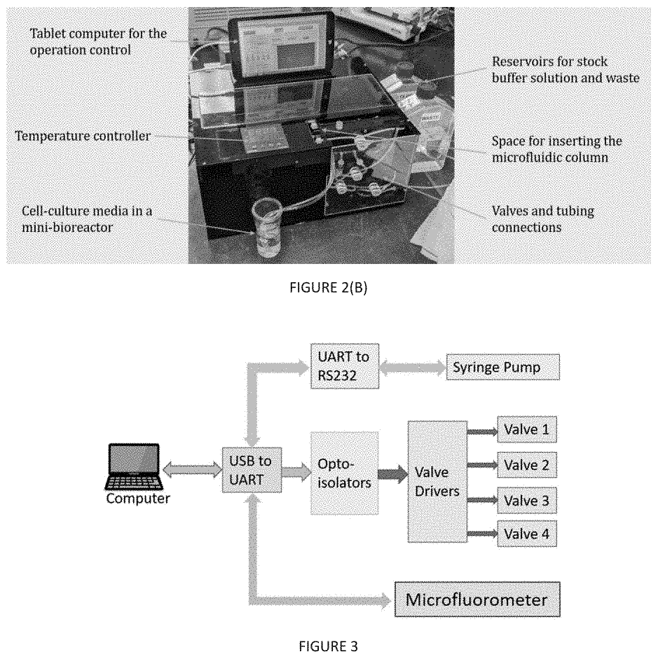

In one embodiment, the sample collection technique utilizes a microdialysis device (e.g., a PIERCE 96-well microdialysis sampler, which comprises two closely positioned cellulose membranes). For example, the commercial microdialysis sampler can be modified by introducing needles into the two holes of the sampler (see, e.g., FIG. 1(B)) and tubes can be connected to the needles. Tubes from the apparatus are attached to the microdialysis device and the device can be introduced into the culture media or the bioreactor (see, e.g., FIG. 2(A) and 2(B)). As will be described herein, the tubes are communicatively connected to buffer solution, which passes through the microdialysis device, allowing glucose to diffuse into the buffer from the culture media or bioreactor. It should be appreciated that other sampling devices are contemplated, as readily understood by the person skilled in the art including, but not limited to, dead-end samplers and dialysis tubing loops.

An advantage of the present device is that the amount of glucose diffusing from the culture media or bioreactor into the buffer flowing through the tubes depends on (i) the flow rate of the buffer, (ii) the diffusion surface area available in the microdialysis device, or (iii) both (i) and (ii). With regards to flow rate, the higher the flow rate, the less glucose that is collected in a fixed volume of buffer and vice-versa. With regards to the available diffusion surface area, the lower the diffusion surface area available, the less glucose that is collected in a fixed volume of buffer and vice-versa. For the measurement of higher concentrations of glucose, the flow rate is preferably increased or the available diffusion surface area decreased, or both, allowing less glucose to diffuse. Flow rate changes and/or diffusion surface area adjustment can be used so that the fluorescence changes remain in a linear range of detection. This is discussed and verified below.

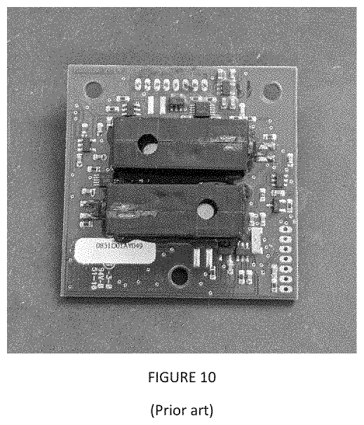

In one embodiment, a fluorimeter, e.g., a microfluorometer, is used for the fluorescence measurements. An example of a microfluorimeter is shown in FIG. 10. The small size of a microfluorometer makes it readily integrable in the apparatus described herein. It should be appreciated by the person skilled in the art that the microfluorometer can comprise just one fluorescence measuring channel, or multiple fluorescence measuring channels (e.g., like in FIG. 10), wherein multiple measurements can be occurring simultaneously. In a preferred embodiment, the microfluidic column, specifically the portion comprising the fluorescently labeled resin, is positioned flush with the microfluorimeter to facilitate fluorescence measurement (not shown). Although reference is made to a microfluorimeter, which is integrable with the portable device described herein, it should be appreciated that any device capable of detecting fluorescence can be used including, but not limited to, traditional fluorometers, minifluorometers, and fiber optics, as readily understood by the person skilled in the art.

Advantageously, the apparatus described herein automates the measurement process. Broadly, the flow of buffer and sample through the apparatus is controlled by a syringe pump. The direction of the flow is controlled by four 2-way pinch valves. The pump, valves, and the fluorometer are interfaced with a computer using a control circuit that also has the necessary drivers for the components. FIG. 2(A) is a schematic of one embodiment of the apparatus, illustrating the connections of the tubing and valves in the apparatus. It should be appreciated that the embodiment shown in FIG. 2(A) is just one possible arrangement and that others are readily envisioned by the person skilled in the art. In the apparatus, a syringe pump (e.g., New Era NE-500) can be used which is the primary source of all fluid circulation within the apparatus. Four pinch valves (e.g., Cole-Parmer Masterflex 3-way pinch valves) can control the direction of the flow within the apparatus represented by V1, V2, V3, and V4. As described hereinabove, a Pierce 96-well Microdialysis device can be modified as shown in FIG. 1(B) to connect to the apparatus to collect the glucose or other analyte diffused from the culture media into the buffer, e.g., Phosphate Buffer Saline (PBS), circulated inside the microdialysis device. The glucose diffused to the PBS is collected in the tube connecting T2 and V4 referred to herein as the “sample tube” which has a dead volume of approximately 1 mL. The check valve can be used to block the leakage of the collected sample into the tubing connecting V2 and T2. For constant supply of the PBS, an external reservoir can be included and connected to the syringe pump through V1, although preferably it is stored outside the apparatus so that the apparatus remains more compact and portable. The PBS is discarded to a waste vessel after washing the tubes and passing through the sensor column. All components except the stock PBS reservoir and the waste bottles can be packed in a compact housing of the apparatus and the microdialysis device is connected to the apparatus via tubes at the needles. An embodiment of the apparatus is shown in FIG. 2(B).

As shown in FIG. 2(A), the microdialysis device is shown as being positioned on its own loop that can be isolated from the flow of liquids from the syringe pump to the waste container. It should be appreciated that alternative arrangements are envisioned. For example, the apparatus can be arranged such that the microanalysis device is positioned elsewhere, for example, between V2 and T2, with valves associated with the entry and exit tubes into the microdialysis device (not shown).

The apparatus can further comprise a control circuit that includes the circuitry for the communication between the computer and the electrical components in the apparatus, and the drivers for the pinch valves as shown in the block diagram in FIG. 3. The FTDI chip FT232RT was used for USB to serial UART communication. The chip SP213E (Sipex Corporation) has been used to convert the signals from the FT232RT chip to RS232 voltage levels and communicate to the syringe pump. The pinch valves can be solenoid valves. The driver circuit provides 300 mA to the solenoid to activate it initially and then decreases it to 20 mA which is enough to hold it in the activated state. The chip IC-GE from IC Haus can be used in the valve driver circuit where the setting and the holding current can be set with external resistors. Being inductive loads, the solenoids tend to generate switching spike voltages and affect the communication, hence an opto-isolator chip PS2802 (California Eastern Laboratories) can optionally be placed before the valve drivers. The syringe pump, pinch valves, and the air pump all are preferably rated 12V and hence, a 12V power supply module (Advanced Energy XLB-01) can be employed to power all the components.

All the functionalities of the device can be controlled using a program, for example, as developed in LabView. Communication to the FT232RT chip via USB and all the electrical components can be accessed independently. A manual mode can be used for debugging and calibration purposes. The program controls the cycles for automatic sample collection and fluorescence measurement, as well as processes the obtained fluorescence data and finds the peak fluorescence amplitude for a sample which can be used to estimate the glucose concentrations.

In one embodiment of the apparatus, each measurement cycle comprises sample collection and fluorescence measurement, wherein a series of flows is directed to different sections of the tubing in the apparatus. During the sample collection, the direction of flow of buffer is as follows:

Syringe pump→V1→V2→T1→Microdialysis device→V3→T2→Sample tube→V4→Waste

The flow rate in this step is referred to as the “sampling flow rate,” which determines the amount of substrate (e.g., glucose) that can diffuse to the buffer inside the microdialysis device and is available to bind to the binding protein (e.g., GBP) in the microfluidic column in the subsequent measurement step. For different concentration ranges of the sample, this flow rate can be varied to fit into the linear range of detection, as described herein. The substrate-containing buffer is collected in the sample tube prior to introduction to the microfluidic column. In the measurement step, the buffer, e.g., PBS, direction of flow is as follows:

Syringe pump→V1→V2→T2→Sample tube→V4→Sensor→Waste

In the measurement step the substrate-containing buffer is introduced to the microfluidic column (i.e., sensor) for measurement. When the substrate-containing buffer passes through the microfluidic column, the substrate binds to the binding protein and increases in fluorescence can be observed, wherein the increase in fluorescence is a function of the concentration of substrate bound to the binding protein. Subsequent to the binding of the substrate-containing buffer to the binding protein, continued introduction of buffer to the column elutes the binding protein until the fluorescence returns the fluorescence value prior to substrate-containing buffer introduction (the baseline). The flow rate in this step is preferably kept constant for fast elution of the binding protein after measurement without creating back-pressure inside the tubes. After the binding-unbinding cycle, the channel is ready to be used again for the next measurement cycle. Each cycle takes 10-12 minutes depending on the sampling flow rate.

Other cycles include, but are not limited to, priming and washing the tubes:

Syringe pump→V1→V2→T1→Microdialysis device→V3→Waste

refilling the syringe with buffer (e.g., PBS), and emptying the dialysis tube. To empty the dialysis tube, an air pump (not shown, but an air pump can be connected to the apparatus at three-way connector T1 and the air egressed using V3 to waste) is used to flush the inside of the microdialysis device so that it remains empty until new substrate-containing buffer is taken in the next cycle. This stops any diffusion of the substrate into the microdialysis device when the sample is not being taken.

It should be appreciated that for estimations of an unknown concentration, a calibration curve is prepared using known concentrations of glucose as discussed further below and as readily understood by the person skilled in the art. Using the information provided herein, a flow rate should be ascertained that ensures that the unknown concentration falls within the linear range of concentrations. The adjustment of flow rates will allow the measurement of glucose in a range over a broad range from about 1 μM to 200 mM of concentration. Statistical and machine learning techniques can also be applied to the calibration data for better estimation accuracy.

Another variation of the microfluidic column sensor is to directly integrate the column with the fluorometer. Instead of columns, the sensors can also be packed in microwells with a diameter close to the diameter of the LED light source in the microfluorometer. This can minimize the amount of immobilized protein biosensor needed and lead to further miniaturization. Alternatively, the microfluidic column can be illuminated all along its length with an LED optic fiber light engine instead of at a single point.

In instances where the concentration of the analyte in the sample is low, repeated pumping/cycling of the sample solution through the microfluidic column, without immediate elution, can enhance the collected signal as more analyte is bound to the protein biosensor.

In a second aspect, a method of measuring the concentration of a substrate in a sample solution is described, said method comprising:
(a) inserting a sampling device into a sample comprising said substrate;
(b) flowing buffer through the sampling device at a flow rate, wherein the substrate diffuses from the sample into the buffer to yield a substrate-containing buffer;
(c) introducing the substrate-containing buffer to a microfluidic column, wherein the substrate binds to a binding protein and an increase in fluorescence occurs; and
(d) measuring the fluorescence using a fluorometer.
   In one embodiment, an apparatus comprising the sampling device, the microfluidic column, and the fluorometer, as described herein, is used to measure the concentration of the substrate in the sample solution. The method can further comprise at least one of: determining the concentration of the substrate in the sample solution using the measured fluorescence and a calibration curve, introducing additional buffer to the microfluidic column to elute the binding protein; or both. in one embodiment, the method further comprises introducing additional buffer to the microfluidic column to elute the binding protein and steps (a)-(d) can be repeated using a different sample solution. The method can be repeated at least 20, at least 30, or preferably at least 40 times using the same microfluidic column.

Advantageously, the glucose monitoring apparatus described herein is a fully automated and continuous binding protein-based glucose sensor. The sample collection and the measurement techniques allow the convenient extension of the detection range and substantially eliminates the possibility of contamination. The implemented technique generalizes the measurement process to the extent that same glucose monitoring device can be used for the measurement of other substrates in bioprocesses including, but not limited to, Glutamine, Branch-Chained Amino Acids (BCAA), and Leucine, by packing the microfluidic column with the corresponding binding proteins. Apart from the fermentation or other bioprocess-related applications, the glucose monitoring device could be used as a non-invasive glucose monitoring device for humans by replacing the microdialysis device with a sampling head or wearable patch, making this a highly versatile glucose monitoring platform.

The features and advantages of the invention are more fully illustrated by the following non-limiting examples, wherein all parts and percentages are by weight, unless otherwise expressly stated.

### Example 1

### A. Methods and Materials

H152C Glucose Binding Proteins (GBP) were expressed, purified, and fluorescently labelled with BADAN (6-bromoacetyl-2-dimethylaminonaphthalene). The labelled GBP was immobilized with Ni-NTA agarose beads. For immobilization, the microfluidic column devised by Andar et al. [Andar et al., 2019] was used. With some modifications to the original design, six layers were cut according to the required dimensions using CO₂ laser printer and then joined together by ethanol solvent bonding method [Andar et al., 2019]. A 20 μm PTFE frit of 1.5 mm thickness was inserted at the outlet prior to packing the column. Ni-NTA beads were packed into the column first and then fluorescently labelled GBP was circulated through the column for binding. After packing, a similar frit was inserted at the inlet. The frits keep the beads in place and stabilize them while the sample flows through it making fluorescence readings more consistent. A packed microfluidic column is showed in FIG. 1(A).

For fluorescence measurements, the microfluorometer reported by Sardesai et al. [Sardesai et al., 2015] was used. The channel in the microfluidic column was illuminated with violet light (wavelength=405 nm) and modulated with a 10 kHz in-phase signal for the measurement. The microfluorometer and the microfluidic columns are placed inside the enclosed setup so that the channel in the column sits directly above, or flush with, one of the fluorescence measuring channels on the microfluorometer.

In one embodiment of the apparatus, each measurement cycle comprises sample collection and fluorescence measurement. During the sample collection, the direction of flow of buffer is as follows:

Syringe pump→V1→V2→T1→Microdialysis device→V3→T2→Sample tube→V4→Waste

The flow rate in this step is referred to as the “sampling flow rate,” which determines the amount of glucose that can diffuse to the buffer inside the microdialysis device and is available to bind to the binding protein (e.g., GBP) in the microfluidic column in the subsequent measurement step. For different concentration ranges of the glucose sample, this flow rate is varied to fit into the linear range of detection, as described herein. The sample is collected in the sample tube prior to introduction to the microfluidic column. In the measurement step, the buffer, e.g., PBS, direction of flow is as follows:

Syringe pump→V1→V2→T2→Sample tube→V4→Sensor→Waste

In the measurement step the sample is introduced to the microfluidic column (i.e., sensor) for measurement. When the glucose sample passes through the microfluidic column, it binds to the binding protein and increases in the fluorescence can be observed, wherein the increases in fluorescence is a function of the concentration of glucose bound to the binding protein. Subsequent to the binding of the glucose to the binding protein, continued introduction of buffer to the column elutes the binding protein until the fluorescence returns the fluorescence value prior to glucose sample introduction (the baseline). The flow rate in this step is preferably kept constant at 1 mL/min for fast elution of the binding protein after measurement without creating back-pressure inside the tubes. After the binding-unbinding cycle, the channel is ready to be used again for the next measurement cycle.

### B. Experiments

a. Proof of Concept and Sensor Characterization

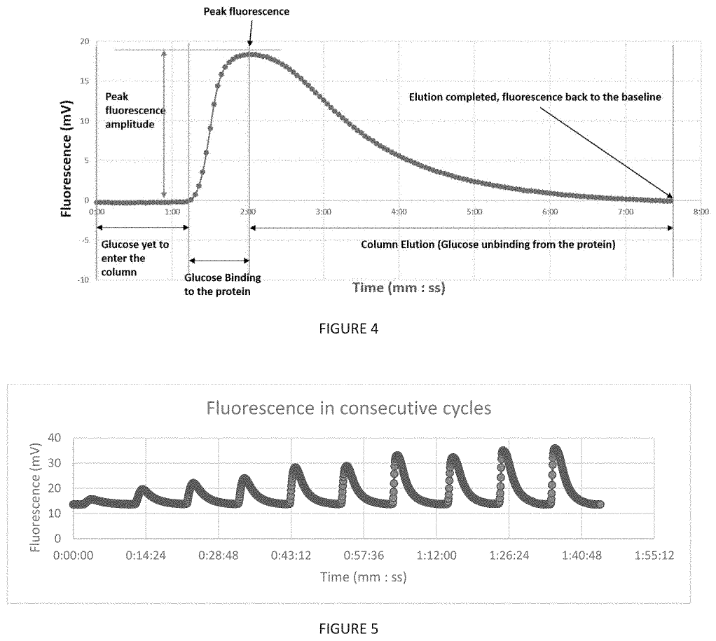

The binding and unbinding cycle can be understood better from FIG. 4 which shows the fluorescence readings in a measurement cycle of a sample solution having a glucose concentration of 40 mM in LB media, with a sampling flow rate of 2 mL/min. The figure shows the rise of fluorescence from the baseline over time in a measurement cycle. In the first section of the curve, the sample is yet to enter the column. The increasing fluorescence as the glucose passes through the column is seen in the following section of the curve and the last section shows the glucose unbinding from the GBP while eluting buffer passes through the column. The peak value of the fluorescence gives the peak fluorescence amplitude which is the difference between the peak value and the base fluorescence value.

An example of consecutive cycles using the same column, using different concentrations of glucose, is shown in FIG. 5, wherein the repeated binding and unbinding of the glucose to the GBP is easily visualized.

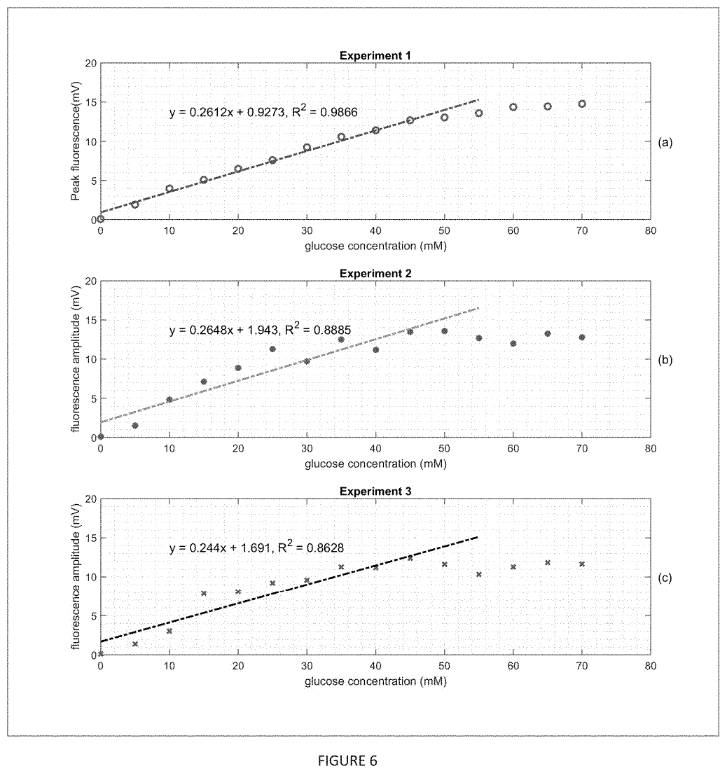

Experiments were conducted to find the linear range of detection. In the first set of experiments, three separate experiments were conducted with glucose concentrations of 0 to 50 mM with steps of 5 mM. The sampling flow rate was 2 mL/min. The experimental glucose solution was held in a bioreactor and the microdialysis device was immersed therein as shown in FIG. 2(B). The first experiment was conducted with a new microfluidic column packed with freshly immobilized GBP, whereas the second experiment was conducted using the same microfluidic column except the GBP has gone through dozens of binding-unbinding cycles. For the third experiment, a different microfluidic column packed with GBP immobilized fabricated on a separate occasion was used. The fluorescence amplitudes for different glucose concentrations are presented in FIG. 6.

As seen in FIG. 6, Experiment 1, the peak fluorescence amplitude for different concentrations is highly correlated to the concentration (R²=0.9866). Experiment 2 in FIG. 6 shows a little degradation in performance with R²=0.8885, which could be attributed to the unfolding of the protein to some extent after several binding-unbinding cycles. Experiment 3 in FIG. 6 has a lower correlation with R²=0.8628, possibly due to some errors in the measurement of a few samples. Even though the experiments were conducted on separate occasions, and Experiment 3 having a completely different microfluidic column, the slopes are found to be very much similar with a low standard deviation of 0.0091. The mean slope was calculated to be 0.2567 mV/mM.

b. Extension of Detection Range by Sampling Flow Rate Variation

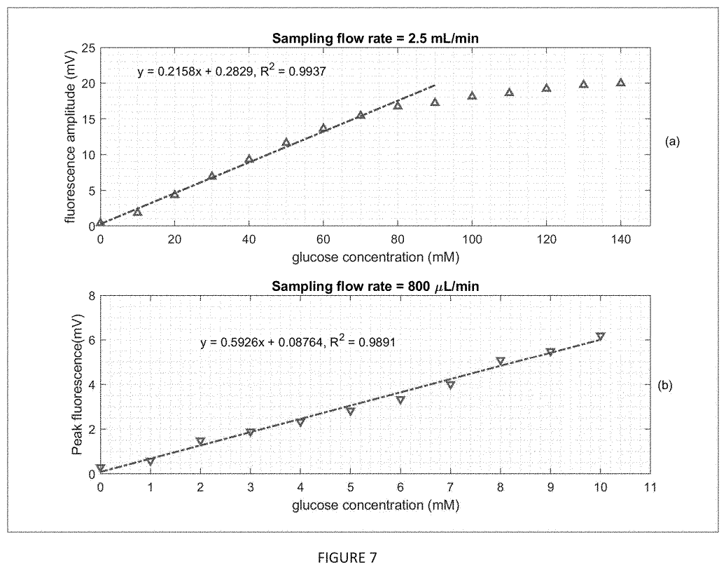

One of the major achievements from the implemented sample collection technique is the extension of the detection range. By varying the sampling flow rate, the amount of glucose allowed to diffuse to the buffer can be controlled. For samples with high glucose concentrations, the flow rate can be increased allowing less glucose to diffuse into the microdialysis device and vice versa. Flow rate changes can be used so that the fluorescence changes remain in a linear range of detection. This was confirmed by a second set of experiments conducted with higher samples having a higher glucose concentration, up to 140 mM, and with lower concentrations, up to 10 mM with sampling flow rates were 2.5 mL/min and 800 μL/min, respectively. The fluorescence amplitudes for different concentrations in this experiment is showed in FIG. 7.

As seen in FIG. 7, the linear range was extended up to 80 mM with an increase in the sampling flow by 0.5 mL/min (relative to the experiments of FIG. 6). Extending the detection range reduced the slope to 0.2158 mV/mM. The slope increased to 0.5926 mV/mM when the sampling flow rate was 800 μL/min with a targeted linear range of detection of 0 to 10 mM glucose. In the above experiments, all the sample solutions were prepared by dissolving dextrose powder (purchased from Sigma-Aldrich) in LB media.

Although not shown, the available diffusion surface area of the microdialysis device can be used to adjust the amount of glucose allowed to diffuse to the buffer, which, like the flow rate, can be used to extend the detection range.

c. Sensor Stability and Storage

The sensor stability and the reversibility of the glucose binding to the GBP were tested extensively in the above experiments. A total of six columns were tested to withstand at least 40 binding-unbinding cycles, each demonstrating a consistent response, after which the protein unfolded to a higher extent and a stable base fluorescence value couldn't be achieved.

In all cases, after the expression and purification of GBP, it was stored at −80° C. without labelling. The GBP used in the first two experiments from the first set of experiments was labelled after 30 days of storage at −80° C. After immobilization of the GBP and packing, the microfluidic columns were stored at 4° C. The second experiment was conducted after 30 days from the first experiment using the same column stored at 4° C. The GBP used in experiments shown in FIG. 7 was labelled after 90 days of storage at −80° C. (with sampling flow rate=800 μL/min), and 120 days at −80° C. (with sampling flow rate=2.5 mL/min). Attempts were also made to store labelled proteins at −80° C., however, it was found to unfold the protein and show inconsistent fluorescence response, likely because of protein damage.

### C. Results and Discussion

This example introduces the construction, operation, and functionality of the glucose monitoring device described herein. The key achievement of this glucose monitoring system is the full automation of a continuous monitoring process. In addition, the sample collection and measurement technique which conveniently allows the extension of the detection range also eliminates the possibility of contamination.

### Example 2

### (b)

A biosensor comprising glutamine binding protein (QBP) was prepared by immobilizing S179C QBP via an affinity tag (e.g., Hexa-histidine tag) on the surface of microbeads (e.g. Ni-NTA dextran). The immobilized protein was manually packed in a 27 μL PMMA microfluidic column having a 20 μm frit at the outlet. A pumping system comprising 2 syringe pumps, a pinch valve and required tubing was attached to the microfluidic column system and the fiber optic/fluorometer was placed flush against the column's bottom layer for fluorescence measurements.

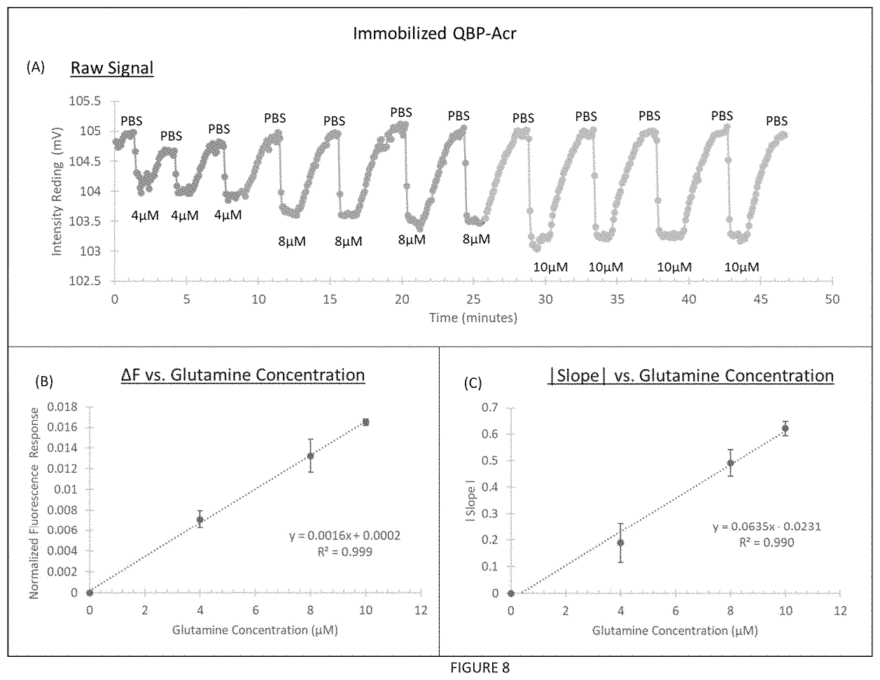

Using optimized fiber optic position and flow rate conditions, the pumping system was used to introduce alternating PBS and micromolar glutamine to the microfluidic biosensor column. FIG. 8 illustrates the resulting on-line fluorometer signal measured from the QBP microfluidic column. The QBP biosensor showed a response time of approximately 15 seconds when exposed to varying micromolar concentrations of glutamine (FIG. 8(A)). The reversibility time ranged from 1 to 3 minutes, allowing for reusability of the biosensor. Increasing the glutamine concentration resulting in increasing normalized intensity signal change (ΔF). When ΔF and corresponding (−) slope were compared to micromolar glutamine concentrations, a significant positive linear relationship (r≥0.995, p≤0.005) was observed, having the trend [y=(0.002±3.733×10⁻⁵) x] and [y=(0.064±0.005)×+0.023±0.030)] respectively (FIGS. 7(B) and 7(C)). Overall, packing immobilized QBP into a microfluidic chromatography column also permits analyte detection reproducibility as well as biosensor reusability.

### Example 3

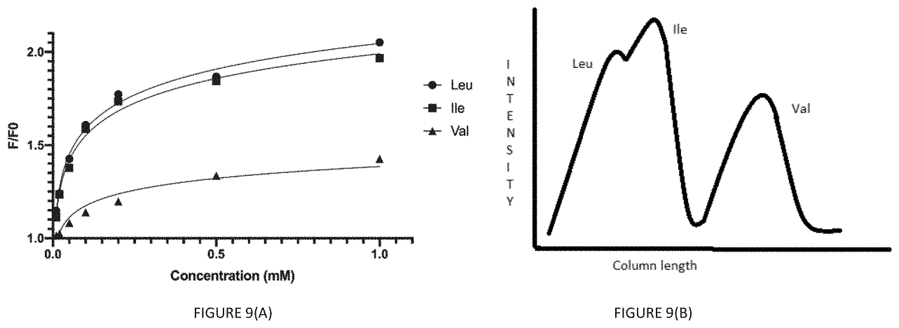

Microfluidic column sensors are also useful when the biosensor detects more than one analyte but with different binding affinities. For example, a branched-chain amino acid binding protein can recognize leucine, isoleucine and valine with K_{d}'s of 58, 68 and 135 μM, respectively. This is shown in FIG. 9, where a mixture of these amino acids can be separately detected over the length of the microfluidic column.

### Example 4

### Calibration Curves

Calibration curves are specific to the flow rate. Accordingly, the data points in a calibration curve should be obtained at the same flow rate.

Initially, the calibration curves are obtained using many different microfluidic columns at various flow rates in order to determine consistent linear ranges of detection at different flow rates.

If the concentration range of the experimental solution is known (e.g., in a cell culture with a known initial glucose concentration, or transdermal samples from humans), the preferred flow rate may be known, based on previously obtained data, to fit the known range of concentrations in the linear region. Two to four known concentrations within this range can be used as calibration samples to obtain a calibration curve. The calibration curve (i.e., the slope and intercept of the linear region) found from these 2-4 points will be used to estimate the glucose concentration within this range.

For an unknown concentration, the flow rate can be set to an approximately median value from all the tested flow rates initially. If the fluorescence response is found in the saturation region, the flow rate can be increased (to a flow rate whose calibration curve is available) and a second iteration performed to check if it fits in the linear range. If the initial response is found to be very low, the flow rate can be reduced (again, to a flow rate whose calibration curve is available) and another iteration performed. After a few such iterations, the flow rate can be determined that accommodates the new experimental concentration in a linear range. The corresponding calibration curve is then readily used to estimate the concentration. Here, the statistical and machine learning techniques can be used to minimize the number of iterations.

In summary, the calibration curve for a known range is obtained right before the measurement using the same column and a few calibration samples. For fully unknown concentrations, the calibration curve is chosen from the available calibration curves.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Brown, Sheniqua R. Fluorescently Labeled Periplasmic Binding Proteins as Optical Biosensors for Detecting Micromolar Levels of Glucose and Glutamine in Biomedical & Bioprocess Applications. University of Maryland, Baltimore County, 2019.

Cuneo, Matthew J., et al, “The crystal structure of a thermophilic glucose binding protein reveals adaptations that interconvert mono and di-saccharide binding sites.” Journal of molecular biology 362.2 (2006): 259-270.

Ge, Xudong, et al. “Genetically engineered binding proteins as biosensors for fermentation and cell culture.” Biotechnology and bioengineering 84.6 (2003): 723-731.

Ge, Xudong, Leah Tolosa, and Govind Rao. “Dual-labeled glucose binding protein for ratiometric measurements of glucose.” Analytical chemistry 76.5 (2004): 1403-1410.

Ge, Xudong, Yordan Kostov, and Govind Rao. “Low-cost noninvasive optical CO2 sensing system for fermentation and cell culture.” Biotechnology and bioengineering 89.3 (2005): 329-334.

Ge, Xudong, et al: “Comparing the performance of the optical glucose assay based on glucose binding protein with high-performance anion-exchange chromatography with pulsed electrochemical detection: efforts to design a low-cost point-of-care glucose sensor.” Journal of diabetes science and technology 1.6 (2007): 864-872.

Ge, Xudong, Govind Rao, and Leah Tolosa. “On the possibility of real-time monitoring of glucose in cell culture by microdialysis using a fluorescent glucose binding protein sensor.” Biotechnology progress 24.3 (2008): 691-697.

Hellinga, Homme W., and Jonathan S. Marvin. “Protein engineering and the development of generic biosensors.” Trends in biotechnology 16.4 (1998): 183-189.

Kostov, Yordan, et al. “Portable system for the detection of micromolar concentrations of glucose.” Measurement Science and Technology 25.2 (2014): 025701.

Kubo, Izumi. “Potentiometric phosphate-sensing system utilizing phosphate-binding protein.” Analytical and bioanalytical chemistry 372.2 (2002): 273-275.

Lucas F. Ribeiro, Vanesa Amarelle, Liliane F. C. Ribeiro, Maria-Eugenia Guazzaroni, “Converting a Periplasmic Binding Protein into a Synthetic Biosensing Switch through Domain Insertion”, BioMed Res. Intl., vol. 2019, Article ID 4798793, 15 pages, 2019. https://doi.org/10.1155/2019/4798793

Naal, Zeki, et al. “Amperometric TNT biosensor based on the oriented immobilization of a nitroreductase maltose binding protein fusion.” Analytical chemistry 74:1 (2002): 140-148.

Sardesai, Neha, Govind Rao, and Yordan Kostov. “Versatile common instrumentation for optical detection of pH and dissolved oxygen.” Review of Scientific Instruments 86.7 (2015): 074302.

Saxl, Tania, et al “A fluorescence lifetime-based fibre-optic glucose sensor using glucose/galactose-binding protein.” Analyst 136.5 (2011): 968-972.

Tian, Yaji, et al. “Stricture-based design of robust glucose biosensors using a Thermotoga maritima periplasmic glucose-binding protein.” Protein Science 16.10 (2007): 2240-2250.

Tiangco, Cristina, et al. “Fluomphore-labeled bioengineered glucose binding protein for measurement of transdermal glucose,” Acta Manilana 64 (2016): 25-32.

Tolosa, Leah, et al. “Glucose sensor for low-cost lifetime-based sensing using a genetically engineered protein.” Analytical biochemistry 267.1 (1999): 114.

Tolosa, Leah, Xudong Ge, and Govind Rao. “Reagentless optical sensing of glutamine using a dual-emitting glutamine-binding protein.” Analytical biochemistry 314.2 (2003): 199-205.

What is claimed is:

1. An apparatus for measuring the concentration of a substrate, said apparatus comprising:
a sampling device;
a microfluidic column device comprising a microfluidic column which comprises a solid support comprising immobilized binding protein, wherein the sampling device is communicatively connected to the microfluidic column; and
a fluorometer.

2. The apparatus of claim 1, wherein the microfluidic column has a volume of about 0.1 to about 200 μL.

3. The apparatus of claim 1, wherein the solid support is in the form of beads, spheres, particles, granules, a gel, or a surface.

4. The apparatus of claim 1, wherein the sampling device comprises a microdialysis device.

5. The apparatus of claim 1, wherein the fluorometer is a microfluorometer.

6. The apparatus of claim 1, wherein the microfluidic column is positioned flush with the fluorimeter to facilitate fluorescence measurements.

7. The apparatus of claim 1, wherein the apparatus further comprising at least one of: a syringe pump to circulate fluid within the apparatus; at least one valve to control the flow direction of the fluid within the apparatus; a check valve; a control circuit for communication between a computer and electrical components in the apparatus; tubing for communicative connections; a computer program; a computer; and any combination thereof.

8. The apparatus of claim 1, wherein the substrate is glucose and the binding protein is a glucose binding protein.

9. The apparatus of claim 1, wherein the solid support comprises Ni-NTA agarose beads.

10. A method of measuring the concentration of a substrate in a sample solution, said method comprising:
(a) inserting the sampling device of the apparatus of claim 1 into a sample comprising said substrate;
(b) flowing buffer through the sampling device at a flow rate, wherein the substrate diffuses from the sample into the buffer to yield a substrate-containing buffer;
(c) introducing the substrate-containing buffer to the microfluidic column, wherein the substrate binds to the binding protein and an increase in fluorescence occurs; and
(d) measuring the fluorescence using the fluorometer.

11. The method of claim 10, wherein the microfluidic column is packed with solid support by:
introducing the solid support to the microfluidic column, and
flowing the binding protein to the solid support for a time necessary to effectuate immobilization of the binding protein to the solid support.

12. The method of claim 10, wherein the sample solution is a culture media solution or a bioreactor solution.

13. The method of claim 10, wherein the flow rate within the apparatus is controlled by a syringe pump.

14. The method of claim 10, further comprising determining the concentration of the substrate in the sample solution using the measured fluorescence and a calibration curve.

15. The method of claim 10, wherein (i) the flow rate, a diffusion surface area of the sampling device, or (iii) both (i) and (ii) can be used to determine the amount of substrate that can diffuse into the buffer in the sampling device and is available to bind to the binding protein in the microfluidic column.

16. The method of claim 15, wherein (i) the flow rate, (ii) the diffusion surface area of the sampling device, or (iii) both (i) and (ii) is controlled so that the measured fluorescence remains in a linear portion of a calibration curve.

17. The method of claim 10, wherein the increase in fluorescence is a function of the concentration of substrate bound to the binding protein.

18. The method of claim 10, further comprising (e) introducing additional buffer to the microfluidic column to elute the binding protein.

19. The method of claim 18, further comprising repeating steps (a)-(d) to determine the concentration of substrate in a second sample solution.

20. The method of claim 10, wherein the method is fully automated and continuous.
